(19) United States
(12) Patent Application Publication
(10) Pub. No.: US 2019/0328265 A1
(43) Pub. Date: Oct. 31, 2019

(54) MAGNETIC RESONANCE IMAGE DISPLAY DEVICE AND MAGNETIC RESONANCE IMAGE DISPLAY METHOD

(71) Applicant: SAMSUNG ELECTRONICS CO., LTD., Suwon-si (KR)

(72) Inventors: Sang-young ZHO, Suwon-si (KR); Min-oh KIM, Yongin-si (KR); Dae-ho LEE, Seongnam-si (KR)

(73) Assignee: SAMSUNG ELECTRONICS CO., LTD., Suwon-si (KR)

(21) Appl. No.: 16/478,738

(22) PCT Filed: Jan. 2, 2018
(86) PCT No.: PCT/KR2018/000030

§ 371 Date: Jul. 17, 2019

(30) Foreign Application Priority Data
Jan. 31, 2017 (KR) 10-2017-0014113

(51) Int. Cl. A61B 5/055; G01R 33/54

(52) U.S. Cl. CPC A61B 5/055; G01R 33/54

(57) ABSTRACT

Provided are a magnetic resonance (MR) image display apparatus, and a method of displaying a medical image. The MR image display apparatus includes a display; a processor; and a memory connected to the processor, wherein, the memory stores instructions executed by the processor for: receiving an input regarding a region of interest of an object; obtaining a positioning image for determining a location of the region of interest within a scanner of a magnetic resonance imaging (MRI) apparatus; and displaying, on the display, whether the location of the region of interest included in the positioning image corresponds to a location of an isocenter of the scanner.

### TECHNICAL FIELD

The present disclosure relates to a magnetic resonance (MR) image display apparatus, and a method of displaying a medical image. More particularly, the present disclosure relates to an MR image display apparatus for displaying an image for use in positioning a location of a region of interest (ROI), and an MR image displaying method performed by the MR image display apparatus.

### BACKGROUND ART

Magnetic resonance imaging (MRI) apparatuses are apparatuses for capturing images of an inside of an object by using a magnetic field, and are widely used to accurately diagnose a disease since the MRI apparatuses three-dimensionally show not only bones, but also discs, joints, nerves, ligaments, and hearts at a desired angle.

In order to obtain an MR image of a region of interest (ROI), an operator may first position a to-be-scanned part within a scanner and then obtain a scout image. The scout image may have lower resolution than a final image of the ROI. In addition, according to a scout scan, an image may be obtained within a shorter time period than it takes to obtain a final image. Accordingly, the operator of an MR image display apparatus may check whether the ROI corresponds to a predetermined location within the scanner, by using the scout image obtained before the final image is obtained. The predetermined location may be, for example, an isocenter that is the center point of a gradient magnetic field in x, y, and z axial directions within the scanner.

When the ROI includes extremities including hands, feet, or knees, the scanner of the MRI apparatus may not include a table capable of controlling the ROI to be set at a preset location. In this case, the operator manually positions the ROI such that the location of the ROI corresponds to an isocenter within the scanner.

### DESCRIPTION OF EMBODIMENTS

### Technical Problem

When a user manually positions the location of a region of interest (ROI) into a scanner of a magnetic resonance imaging (MRI) apparatus, information about the location of the ROI is provided to the user in real time, and thus, a time period taken to position the ROI is reduced.

### Solution to Problem

According to an aspect of an embodiment, a magnetic resonance (MR) image display apparatus includes a display; a processor; and a memory connected to the processor, wherein, the memory stores instructions executed by the processor for: receiving an input regarding a region of interest of an object; obtaining a positioning image for determining a location of the region of interest within a scanner of a magnetic resonance imaging (MRI) apparatus; and displaying, on the display, whether the location of the region of interest included in the positioning image corresponds to a location of an isocenter of the scanner.

According to the embodiment, the memory may further store an instruction executed by the processor for obtaining the positioning image while a location of the object within the scanner is being changed.

According to the embodiment, the memory may further store an instruction executed by the processor for overlapping the positioning image with guide information and displaying a result of the overlapping, when a location of a predetermined spot on the region of interest is within a first distance from the isocenter, and the guide information may include at least one base line representing the location of the isocenter, and location information of the region of interest.

According to the embodiment, the location information of the region of interest may include at least one of location information of the predetermined spot with respect to the isocenter and a direction in which the object is to move.

According to the embodiment, the location information of the region of interest may include a degree to which a location of the predetermined spot deviates from the isocenter.

According to the embodiment, the memory may further store an instruction executed by the processor for repeating the obtaining of the positioning image and the displaying of, on the display, whether the location of the region of interest included in the positioning image corresponds to the location of the isocenter.

According to the embodiment, the memory may further store an instruction executed by the processor for stopping the obtaining of the positioning image when the location of the object within the scanner is not changed for a predetermined time period or when it is determined that the location of the region of interest corresponds to the location of the isocenter.

According to the embodiment, the memory may further store an instruction executed by the processor for displaying, by using at least one light-emitting device, whether the location of the region of interest corresponds to the location of the isocenter.

According to the embodiment, the memory may further store an instruction executed by the processor for displaying, by using at least one light-emitting device, at least one of location information of the predetermined spot on the region of interest with respect to the isocenter, a direction in which the object is to move, and a degree to which the location of the predetermined spot deviates from the location of the isocenter.

According to an aspect of another embodiment, a method of displaying a magnetic resonance (MR) image may include: receiving an input regarding a region of interest of an object, obtaining a positioning image for determining a location of the region of interest within a scanner of a magnetic resonance imaging (MRI) apparatus; and displaying, on a display, whether the location of the region of interest included in the positioning image corresponds to a location of the isocenter of the scanner.

### ADVANTAGEOUS EFFECTS OF DISCLOSURE

When a user manually positions the location of a region of interest (ROI) into a scanner of a magnetic resonance imaging (MRI) apparatus, information about the location of the ROI is provided to the user in real time, and thus, a time period taken to position the ROI is reduced.

### BRIEF DESCRIPTION OF DRAWINGS

These and/or other aspects will become apparent and more readily appreciated from the following description of the embodiments, taken in conjunction with the accompanying drawings in which:

FIG. 1 is a block diagram of a magnetic resonance (MR) image display apparatus according to an embodiment of the present inventive concept;

FIG. 2 is a view for explaining a positioning image displayed on the MR image display apparatus of FIG. 1, according to an embodiment;

FIG. 3 is a view for explaining a positioning image displayed on the MR image display apparatus of FIG. 1, according to an embodiment;

FIG. 4 is a view for explaining a positioning image displayed on the MR image display apparatus of FIG. 1, according to an embodiment;

FIG. 5 is a diagram for explaining the MR image display apparatus of FIG. 1 displaying, by using a plurality of light-emitting devices, an indication of whether a location of a region of interest (ROI) corresponds to a location of an isocenter, according to an embodiment;

FIG. 6 is a diagram for explaining the MR image display apparatus of FIG. 1 displaying, by using a plurality of light-emitting devices, a movement distance for moving an object to the location of the isocenter, according to an embodiment;

FIG. 7 is a diagram for explaining the MR image display apparatus of FIG. 1 displaying, by using a plurality of light-emitting devices, a movement velocity for moving the object to the location of the isocenter, according to an embodiment;

FIG. 8 is a diagram for explaining the MR image display apparatus of FIG. 1 displaying, by using a plurality of light-emitting devices, an indication of whether the location of the ROI corresponds to the location of the isocenter, according to an embodiment;

FIG. 9 is a flowchart of a method, performed by the MR image display apparatus of FIG. 1, of displaying a medical image, according to an embodiment;

FIG. 10 is a flowchart of a method, performed by the MR image display apparatus of FIG. 1, of displaying a medical image, according to an embodiment;

FIG. 11 is a flowchart of a method, performed by the MR image display apparatus of FIG. 1, of displaying a medical image, according to an embodiment; and

FIG. 12 is a schematic diagram of a general magnetic resonance imaging (MRI) system.

### MODE OF DISCLOSURE

The present specification describes principles of the present disclosure and sets forth embodiments thereof to clarify the scope of the present disclosure and to allow those of ordinary skill in the art to implement the embodiments. The present embodiments may have different forms.

Like reference numerals refer to like elements throughout. The present specification does not describe all components in the embodiments, and common knowledge in the art or the same descriptions of the embodiments will be omitted below. The term “part” or “portion” may be implemented using hardware or software, and according to embodiments, one “part” or “portion” may be formed as a single unit or element or include a plurality of units or elements. Hereinafter, the principles and embodiments of the present disclosure will be described in detail with reference to the accompanying drawings.

In the present specification, an “image” may include a medical image obtained by a magnetic resonance imaging (MRI) apparatus, a computed tomography (CT) apparatus, an ultrasound imaging apparatus, an X-ray apparatus, or another medical imaging apparatus.

Furthermore, in the present specification, an “object” may be a target to be imaged and include a human, an animal, or a part of a human or animal. For example, the object may include a body part (an organ) or a phantom.

An MRI system acquires an MR signal and reconstructs the acquired MR signal into an image. The MR signal denotes a radio frequency (RF) signal emitted from the object.

In the MRI system, a main magnet creates a static magnetic field to align a magnetic dipole moment of a specific atomic nucleus of the object placed in the static magnetic field along a direction of the static magnetic field. A gradient coil may generate a gradient magnetic field by applying a gradient signal to a static magnetic field and induce resonance frequencies differently according to each region of the object.

An RF coil may emit an RF signal to match a resonance frequency of a region of the object whose image is to be acquired. Furthermore, when gradient magnetic fields are applied, the RF coil may receive MR signals having different resonance frequencies emitted from a plurality of regions of the object. Though this process, the MRI system may obtain an image from an MR signal by using an image reconstruction technique.

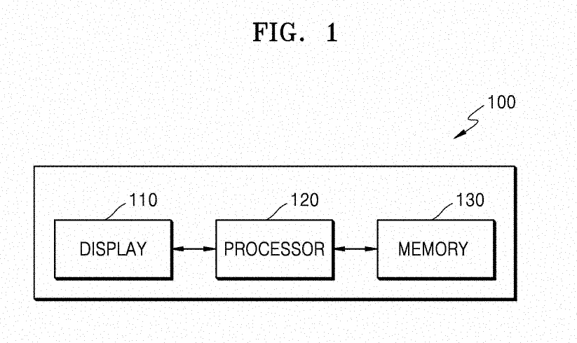

FIG. 1 is a block diagram of an MR image display apparatus 100 according to an embodiment of the present inventive concept.

Referring to FIG. 1, the MR image display apparatus 100 may include a display 110, a processor 120, and a memory 130.

The MR image display apparatus 100 may inform a user of whether a location of a region of interest of an object corresponds to an isocenter of a scanner of an MRI apparatus, before obtaining a medical image of the object.

The MR image display apparatus 100 may be an MRI apparatus. The MR image display apparatus 100 may be an apparatus connected to an MRI apparatus to control the MRI apparatus. For example, the MR image display apparatus 100 may be included in a console for controlling an MRI apparatus.

When the MR image display apparatus 100 is an MRI apparatus, the display 110 may be included in the MRI apparatus. The display 110 may be attached to the MRI apparatus and operate.

The processor 120 according to an embodiment may execute an instruction stored in the memory 130.

The processor 120 may be configured to obtain an MR image, based on MR signal data stored in the memory 130 or MR signal data received from an external device (not shown). For example, the MR signal data may include an MR signal received from a scanner (not shown).

The memory 130 according to an embodiment may store instructions executed by the processor 120.

For example, the memory 130 may store various pieces of data, programs, or applications for driving and controlling the MR image display apparatus 100. A program stored in the memory 130 may include at least one instruction. A program or application stored in the memory 130 may be executed by the processor 120.

The memory 130 may store an instruction of receiving an input for the region of interest of the object. The region of interest of the object may include a part of the object that the user desires to scan.

The memory 130 may also store an instruction for obtaining a positioning image.

A positioning image according to an embodiment may be an image for determining the location of the region of interest within the scanner of the MRI apparatus. The positioning image may be a scout image enabling a user to know the location of the to-be-scanned part of the object when the to-be-scanned part of the object is positioned at an appropriate location.

The location of the region of interest according to an embodiment may be a location of a predetermined spot on the part of the object that the user desires to scan.

The memory 130 may store an instruction for displaying, on the display 110, whether the location of the region of interest included in the positioning image corresponds to a location of the isocenter of the scanner.

The location of the region of interest corresponding to the location of the isocenter may mean positioning the predetermined spot on the region of interest at the isocenter.

The memory 130 may also store an instruction for obtaining a positioning image while the location of the object within the scanner is being changed.

When the location of the predetermined spot on the region of interest is within a first distance from the isocenter, the memory 130 may further store an instruction for overlapping the positioning image with guide information and display a result of the overlapping.

The predetermined spot on the region of interest may be, for example, a spot previously set by a user input. The predetermined spot of the region of interest may include the center of the region of interest. The predetermined spot on the region of interest may be a preset spot for each to-be-scanned part, and may be an optimal location determined based on reference data for the region of interest.

The first distance may be a distance by which the location of the predetermined spot of the region of interest is close to the isocenter. For example, the first distance may be previously set to be 50 mm, and may vary according to embodiments.

For example, the location of the predetermined spot on the region of interest being within the first distance from the isocenter may be the predetermined spot being located within 50 mm from the isocenter.

The guide information may include at least one base line representing the location of the isocenter, and location information of the region of interest.

The at least one base line may include a horizontal line and a vertical line that intersect each other at a spot corresponding to the isocenter within the positioning image. The spot corresponding to the isocenter within the positioning image may be a center of the positioning image.

The location information of the region of interest may include at least one of location information of the predetermined spot with respect to the isocenter and a direction in which the object is to move.

The location information of the region of interest may also include the degree to which the location of the predetermined spot deviates from the location of the isocenter.

The memory 130 may further store an instruction for repeating obtaining the positioning image and displaying, on the display 110, whether the location of the region of interest included in the positioning image corresponds to the location of the isocenter.

When the location of the object within the scanner is not changed for a predetermined time period, the memory 130 may further store an instruction for stopping obtaining the positioning image.

The memory 130 may further store an instruction for displaying, by using at least one light-emitting device, whether the location of the region of interest corresponds to the location of the isocenter.

The at least one light-emitting device may be attached to the MRI apparatus. In detail, the at least one light-emitting device may be attached to a location enabling the user to easily determine whether a light-emitting device emits light while positioning the object.

A light-emitting device according to an embodiment may include, but is not limited to, a light-emitting diode (LED).

The memory 130 may further store an instruction for displaying, by using the at least one light-emitting device, at least one of the location information of the predetermined spot with respect to the isocenter, the direction in which the object is to move, and the degree to which the location of the predetermined spot deviates from the location of the isocenter.

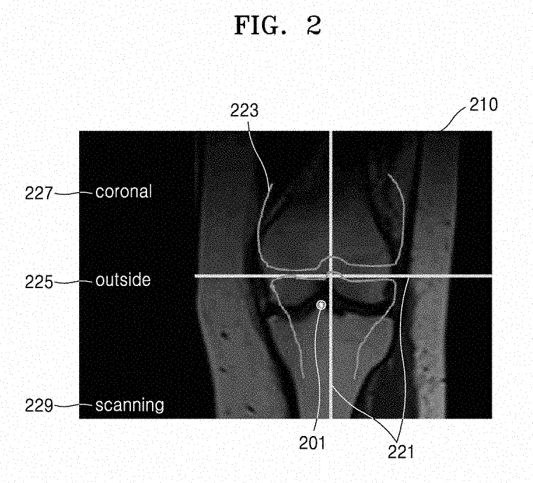

FIG. 2 is a view for explaining a positioning image 210 displayed on the MR image display apparatus 100, according to an embodiment.

The MR image display apparatus 100 may overlap the positioning image 210 with guide information and display a result of the overlapping.

For example, when a location of a predetermined spot 201 on a region of interest is within a first distance from the isocenter, the MR image display apparatus 100 may overlap the positioning image 210 with the guide information and display a result of the overlapping.

The guide information may include at least one base line 221 representing the location of the isocenter, and location information of the region of interest.

For example, the location information of the region of interest may include a FIG. 223 representing the outline of a to-be-scanned part. The location information of the region of interest may also include at least one of location information of the predetermined spot 201 with respect to the isocenter and a direction 225 in which an object is to move.

The FIG. 223 representing the outline of the to-be-scanned part may represent a desirable location of the region of interest.

In detail, the FIG. 223 representing the outline of the to-be-scanned part may represent an outline of the to-be-scanned part when the location of the predetermined spot 201 on the region of interest is positioned at the isocenter.

Referring to FIG. 2, the MR image display apparatus 100 may display the positioning image 210 including at least one of the at least one base line 221 representing the location of the isocenter, the FIG. 223 representing the outline of the to-be-scanned part, the direction 225 in which the object is to move, a plane 227 currently being scanned, and information 229 indicating whether a scan is being performed.

The MR image display apparatus 100 may further display, on the positioning image 210, at least one of information about the to-be-scanned part currently being scanned and information about a scanning pulse sequence.

A user is able to easily position the to-be-scanned part of the object within the MRI apparatus, based on the guide information displayed on the positioning image 210 of the MR image display apparatus 100.

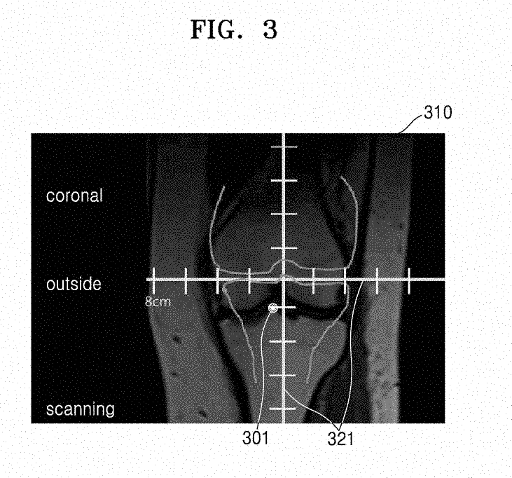

FIG. 3 is a view for explaining a positioning image 310 displayed on the MR image display apparatus 100, according to an embodiment.

The MR image display apparatus 100 may show gradations for representing actual distances, on at least one base line 321 indicating the location of the isocenter, and display the at least one base line 321 on which the gradations are shown.

The MR image display apparatus 100 may also display location information of a predetermined spot 301 with respect to the isocenter, together with the at least one base line 321 indicating the location of the isocenter.

The location information of the predetermined spot 301 with respect to the isocenter may be information numerically representing the degree to which the location of the predetermined spot 301 deviates from the location of the isocenter.

The MR image display apparatus 100 may repeat obtaining the positioning image 310 and displaying the positioning image 310, until it is determined that the location of the predetermined spot 301 corresponds to the location of the isocenter.

For example, when the location of the object within the scanner does not change for a predetermined period of time, the MR image display apparatus 100 may determine that the location of the predetermined spot 301 corresponds to the location of the isocenter.

The MR image display apparatus 100 may determine whether the location of the predetermined spot 301 corresponds to the location of the isocenter, via a special image analysis process. The location of the predetermined spot 301 corresponding to the location of the isocenter may include a case where a distance between the predetermined spot 301 and the isocenter is less than or equal to a predetermined distance.

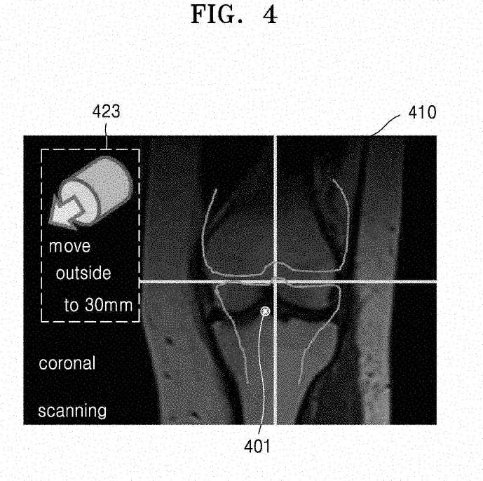

FIG. 4 is a view for explaining a positioning image 410 displayed on the MR image display apparatus 100, according to an embodiment.

The positioning image 410 may include location information 423 of the region of interest, the location information 423 including at least one of location information of the predetermined spot 401 with respect to the isocenter and a direction in which the object is to move.

For example, referring to FIG. 4, the location information of the predetermined spot 401 with respect to the isocenter may be deviating 30 mm from the isocenter, and the direction in which the object is to move may be a direction toward the outside of the scanner. The direction toward the outside of the scanner may be a direction in which the object and the scanner become away from each other.

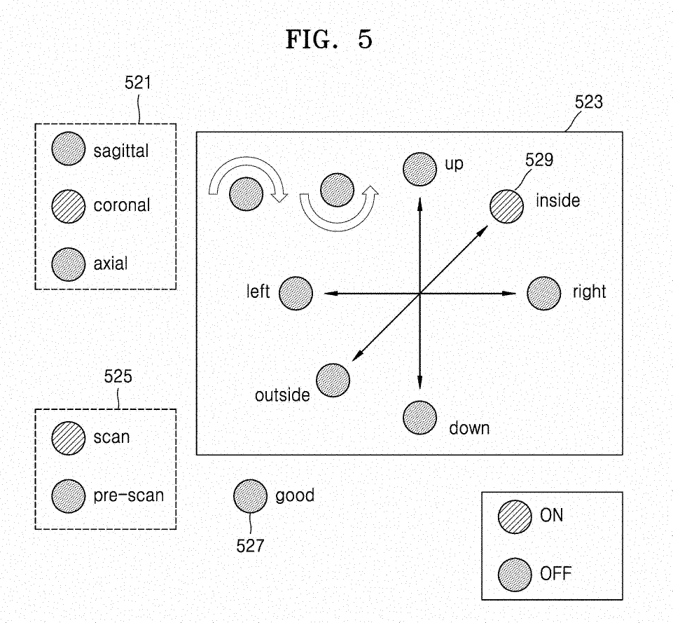

FIG. 5 is a diagram for explaining the MR image display apparatus 100 displaying, by using a plurality of light-emitting devices, whether the location of the region of interest corresponds to the location of the isocenter, according to an embodiment.

Location information 523 of the region of interest may include at least one of location information of the predetermined spot on the region of interest and a direction 529 in which the object is to move.

For example, referring to FIG. 5, the MR image display apparatus 100 may inform a user that the direction 529 in which the object is to move is an inside direction, by turning on a light-emitting device corresponding to the inside direction.

The MR image display apparatus 100 may further display, by using the plurality of light-emitting devices, at least one of a plane 521 currently being scanned and information 525 indicating whether the plane 521 is being scanned.

When it is determined that the location of the region of interest corresponds to the location of the isocenter, the MR image display apparatus 100 may turn on a light-emitting device representing information 527 about whether positioning has been completed.

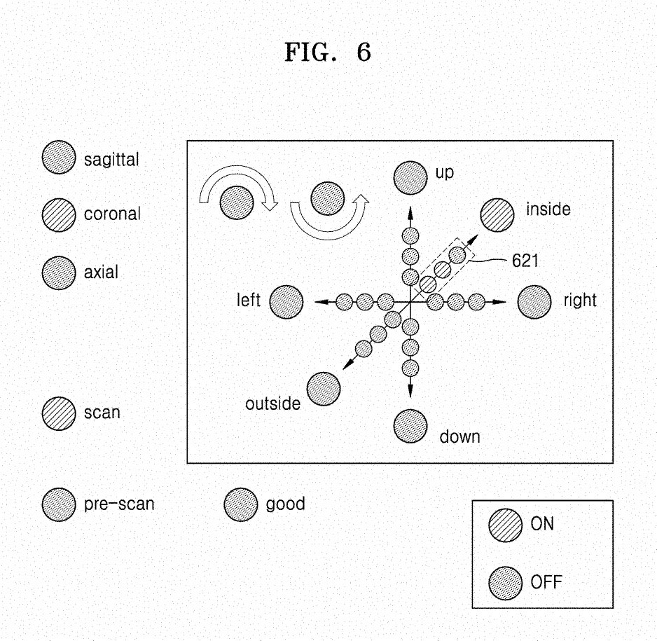

FIG. 6 is a diagram for explaining the MR image display apparatus 100 displaying, by using a plurality of light-emitting devices, a movement distance 621 for moving the object to the location of the isocenter, according to an embodiment.

Referring to FIG. 6, the movement distance 621 for moving the object to the location of the isocenter may be displayed by turning on/off the plurality of light-emitting devices.

For example, when a large number of light-emitting devices from among the plurality of light-emitting devices are turned on, it may mean that the location of the region of interest greatly deviates from the location of the isocenter.

The movement distance 621 may correspond to the degree to which the location of the region of interest deviates from the location of the isocenter.

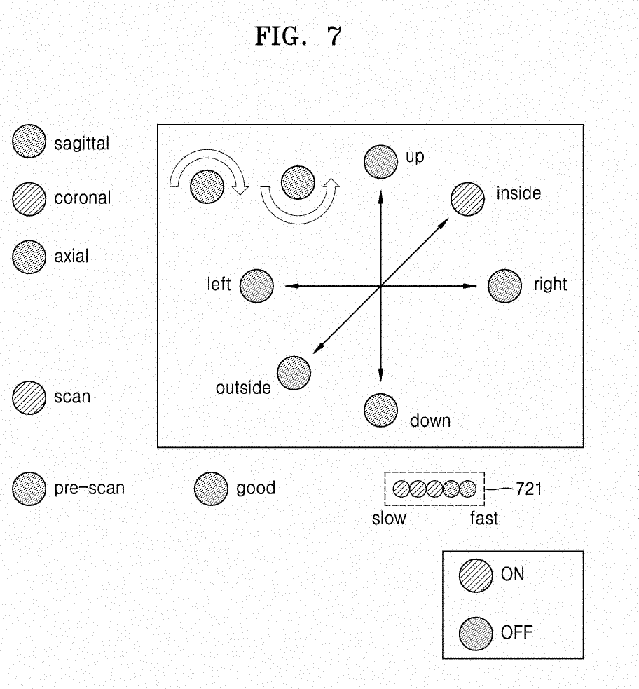

FIG. 7 is a diagram for explaining the MR image display apparatus 100 displaying, by using a plurality of light-emitting devices, a movement velocity 721 for moving the object to the location of the isocenter, according to an embodiment.

The movement velocity 721 for moving the object to the location of the isocenter may be displayed by turning on/off the plurality of light-emitting devices. The movement velocity 721 may correspond to the degree to which the location of the region of interest deviates from the location of the isocenter.

For example, when the location of the region of interest greatly deviates from the location of the isocenter, a user may move the object at a high velocity and position the object, and, when the location of the region of interest slightly deviates from the location of the isocenter, the user may move the object at a low velocity and position the object.

The user may determine a velocity for moving the object in order to position the to-be-scanned part, based on the movement velocity 721 displayed using the plurality of light-emitting devices.

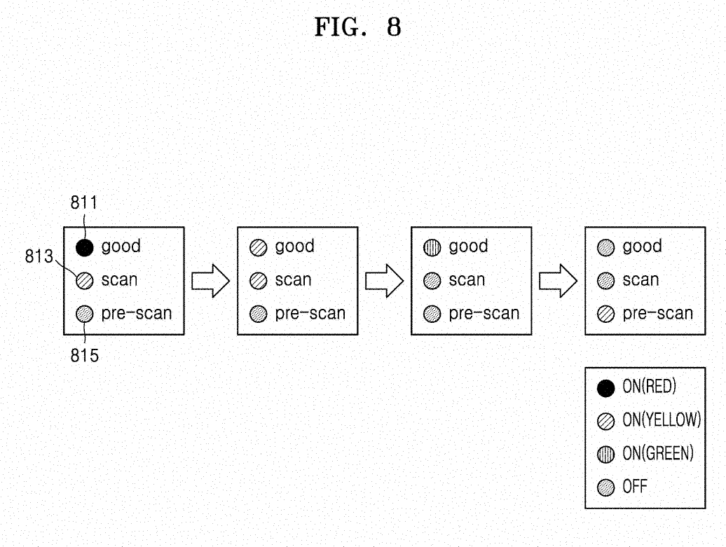

FIG. 8 is a diagram for explaining the MR image display apparatus 100 displaying, by using a plurality of light-emitting devices, whether the location of the region of interest corresponds to the location of the isocenter, according to an embodiment.

Referring to FIG. 8, the plurality of light-emitting devices may include a light-emitting device representing information 811 about whether positioning has been completed, a light-emitting device representing information 813 about whether a scan is being performed, and a light-emitting device representing information 815 about whether a pre-scan is being performed.

The light-emitting device representing the information 811 about whether positioning has been completed may represent a distance between the location of the region of interest and the isocenter by changing the color of emitted light.

Referring to FIG. 8, the light-emitting device representing the information 811 about whether positioning has been completed may be turned on in red when the location of the region of interest greatly deviates from the location of the isocenter. The light-emitting device representing the information 811 about whether positioning has been completed may be turned on in yellow when the location of the region of interest becomes closer to the location of the isocenter. The light-emitting device representing the information 811 about whether positioning has been completed may be turned on in green when it is determined that positioning has been completed.

The MR image display apparatus 100 may start a pre-scan when a scan for obtaining a positioning image has been completed. At this time, the light-emitting device representing the information 813 about whether a scan is being performed may be turned off, and the light-emitting device representing the information 815 about whether a pre-scan is being performed may be turned on.

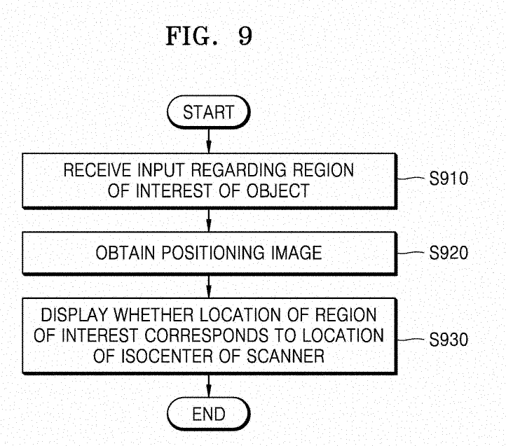

FIG. 9 is a flowchart of a method, performed by the MR image display apparatus 100, of displaying a medical image, according to an embodiment.

In operation S910, the MR image display apparatus 100 may receive an input regarding a region of interest of an object.

In operation S920, the MR image display apparatus 100 may obtain a positioning image. The positioning image may be an image for determining a location of the region of interest within a scanner of an MRI apparatus.

In operation S930, the MR image display apparatus 100 may display whether the location of the region of interest corresponds to a location of an isocenter of the scanner.

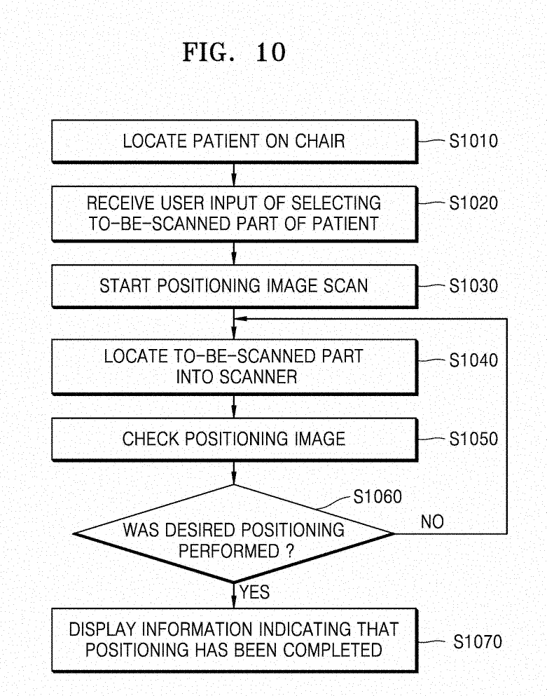

FIG. 10 is a flowchart of a method, performed by the MR image display apparatus 100, of displaying a medical image, according to an embodiment.

In operation S1010, a patient may be located on a chair included in the MR image display apparatus 100.

In operation S1020, the MR image display apparatus 100 may receive a user input of selecting a to-be-scanned part of the patient. According to an embodiment, the MR image display apparatus 100 may receive the user input via a display that receives a touch input.

In response to the user input of selecting the to-be-scanned part of the patient, the MR image display apparatus 100 may start a pre-scan before starting an MR image scan with respect to the to-be-scanned part.

The pre-scan may include a scan of determining whether signal obtainment is possible. The pre-scan may also include a scan for searching for a center frequency. The pre-scan may also include a scan for searching for a transmission gain. The pre-scan may be performed before every image obtainment or may be performed every time an image is obtained with the image obtainment being performed several times.

In operation S1030, the MR image display apparatus 100 may start a positioning image scan.

The MR image display apparatus 100 according to an embodiment may use start a cross-section selective or non-selective RF in order to perform the positioning image scan. According to the positioning image scan, the MR image display apparatus 100 may obtain a single image within one second or may obtain a single positioning image within several seconds.

The positioning image may be a coronal section, a sagittal section, an axial section, or a predetermined section. The section of the positioning image may be previously selected, or may be changed by a user while the user is obtaining the positioning image. The positioning image includes a two-dimensional (2D) or 3D image.

A pulse sequence for obtaining the positioning image may include a gradient echo sequence, a steady-state free precession (SSFP) spin echo sequence, and a burst imaging sequence. Spatio-temporal encoding may be used to obtain the positioning image. The positioning image may be obtained using a Cartesian method and a non-Cartesian method.

To increase temporal resolution of the positioning image, a view sharing technique or a golden angle radial obtaining method may be used when the positioning image is obtained. During re-construction of the positioning image, a sliding window method may be used.

In operation S1040, the user may locate the to-be-scanned part into the scanner of the MR image display apparatus 100.

The user may manually change the to-be-scanned part. A time period during which the to-be-scanned part is manually changed may vary depending on a time period taken to obtain the positioning image.

In operation S1050, the user may check the positioning image displayed on the MR image display apparatus 100.

In operation S1060, the user may determine whether desired positioning was performed, based on the positioning image displayed on the MR image display apparatus 100.

In operation S1060, the MR image display apparatus 100 may determine whether desired positioning was performed, by analyzing the positioning image.

For example, the MR image display apparatus 100 may analyze whether the to-be-scanned part has been positioned, according to a pre-stored program, based on a pre-selected to-be-scanned part (for example, a knee or a wrist).

In operation S1060, the automatic determination by the MR image display apparatus 100 and the determination by the user as to whether desired positioning was performed may be performed simultaneously.

If it is determined in operation S1060 that desired positioning was performed, in operation S1070, the MR image display apparatus 100 may display, to the user, information indicating that positioning has been completed.

The user may perform final positioning by referring to the information displayed on the MR image display apparatus 100.

On the other hand, if it is determined in operation S1060 that desired positioning was not performed, the method may return to operation S1040.

The MR image display apparatus 100 may perform a pre-scan and a localizer scan for a main scan after the final positioning has been completed. When it is determined that the final positioning has been completed, the MR image display apparatus 100 may automatically perform a pre-scan and a localizer scan.

The pre-scan after the final positioning has been completed may include a scan for searching for a center frequency, a scan for searching for a transmission gain, a scan for compensating for receive sensitivity non-uniformity, a scan for searching for non-uniformity of a main magnetic field (B0), and a scan for searching for transmission (B1) sensitivity non-uniformity.

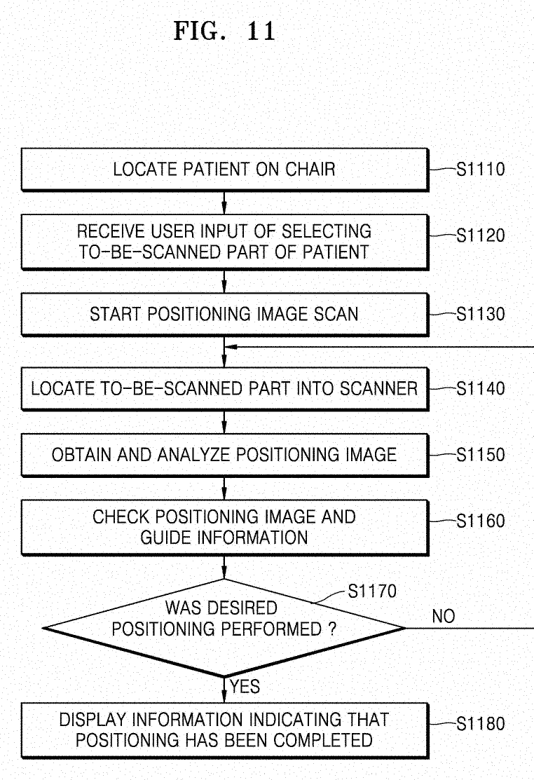

FIG. 11 is a flowchart of a method, performed by the MR image display apparatus 100, of displaying a medical image, according to an embodiment.

Operation S1110, operation S1120, operation S1130, and operation S1140 correspond to operation S1010, operation S1020, operation S1030, and operation S1040 of FIG. 10, respectively, and thus descriptions thereof will be omitted here.

In operation S1150, the MR image display apparatus 100 may obtain a positioning image and analyze the obtained positioning image. The obtained positioning image may be analyzed via machine learning and deep learning.

In operation S1160, a user may check the positioning image and guide information displayed on the MR image display apparatus 100.

Operation S1170 and operation S1180 correspond to operation S1060 and operation S1070 of FIG. 10, respectively, and thus descriptions thereof will be omitted here.

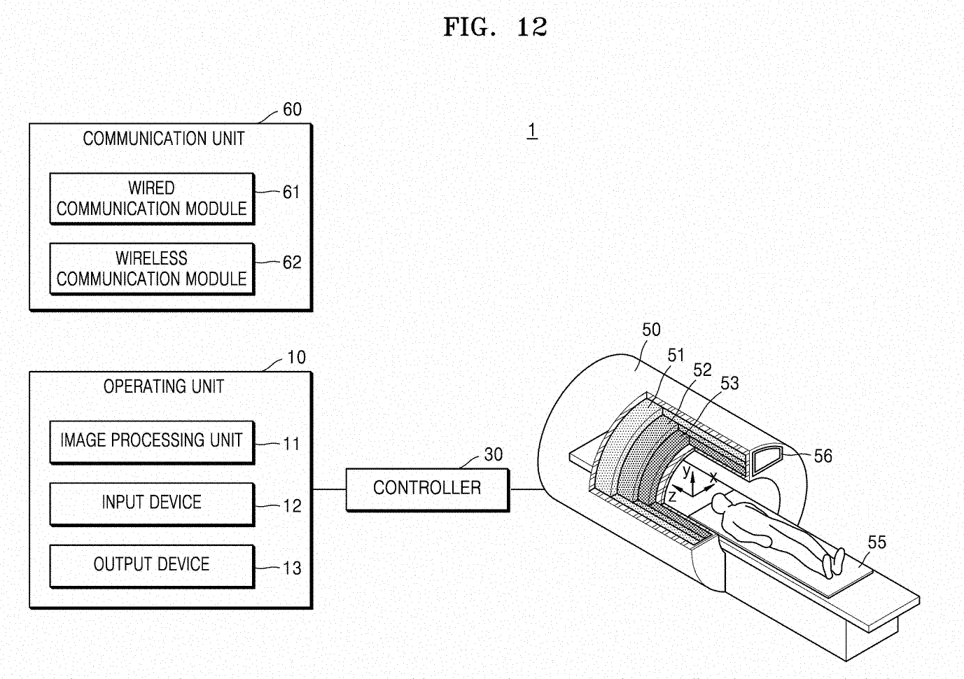

FIG. 12 is a schematic diagram of an MRI system 1.

Referring to FIG. 12, the MRI system 1 may include an operating unit 10, a controller 30, and a scanner 50. The controller 30 may be independently separated from the operating unit 10 and the scanner 50. Furthermore, the controller 30 may be separated into a plurality of sub-components and incorporated into the operating unit 10 and the scanner 50 in the MRI system 1. Operations of the components in the MRI system 1 will now be described in detail.

The scanner 50 may be formed to have a cylindrical shape (e.g., a shape of a bore) having an empty inner space into which an object may be inserted. A static magnetic field and a gradient magnetic field are created in the inner space of the scanner 50, and an RF signal is emitted toward the inner space.

The scanner 50 may include a static magnetic field generator 51, a gradient magnetic field generator 52, an RF coil unit 53, a table 55, and a display 56. The static magnetic field generator 51 creates a static magnetic field for aligning magnetic dipole moments of atomic nuclei of the object in a direction of the static magnetic field. The static magnetic field generator 51 may be formed as a permanent magnet or superconducting magnet using a cooling coil.

The gradient magnetic field generator 52 is connected to the controller 30 and generates a gradient magnetic field by applying a gradient to a static magnetic field in response to a control signal received from the controller 30. The gradient magnetic field generator 52 includes X, Y, and Z coils for generating gradient magnetic fields in X-, Y-, and Z-axis directions crossing each other at right angles and generates a gradient signal according to a position of a region being imaged so as to differently induce resonance frequencies according to regions of the object.

The RF coil unit 53 connected to the controller 30 may emit an RF signal toward the object in response to a control signal received from the controller 30 and receive an MR signal emitted from the object. In detail, the RF coil unit 53 may transmit, toward atomic nuclei of the object having precessional motion, an RF signal having the same frequency as that of the precessional motion, stop transmitting the RF signal, and then receive an MR signal emitted from the object.

The RF coil unit 53 may be formed as a transmitting RF coil for generating an electromagnetic wave having an RF corresponding to the type of an atomic nucleus, a receiving RF coil for receiving an electromagnetic wave emitted from an atomic nucleus, or one transmitting/receiving RF coil serving both functions of the transmitting RF coil and receiving RF coil. Furthermore, in addition to the RF coil unit 53, a separate coil may be attached to the object. Examples of the separate coil may include a head coil, a spine coil, a torso coil, and a knee coil according to a region being imaged or to which the separate coil is attached.

The display 56 may be disposed outside and/or inside the scanner 50. The display 56 is also controlled by the controller 30 to provide a user or the object with information related to medical imaging.

The display 56 may include the display 110 of FIG. 1.

Furthermore, the scanner 50 may include an object monitoring information acquisition unit (not shown) configured to acquire and transmit monitoring information about a state of the object. For example, the object monitoring information acquisition unit may acquire monitoring information related to the object from a camera (not shown) for capturing images of a movement or position of the object, a respiration measurer (not shown) for measuring the respiration of the object, an ECG measurer for measuring the electrical activity of the heart, or a temperature measurer for measuring a temperature of the object and transmit the acquired monitoring information to the controller 30. The controller 30 may in turn control an operation of the scanner 50 based on the monitoring information. Operations of the controller 30 will now be described in more detail.

The controller 150 may control overall operations of the X-ray apparatus 50.

The controller 30 may control a sequence of signals formed in the scanner 50. The controller 30 may control the gradient magnetic field generator 52 and the RF coil unit 53 according to a pulse sequence received from the operating unit 10 or a designed pulse sequence.

A pulse sequence may include all pieces of information required to control the gradient magnetic field generator 52 and the RF coil unit 53. For example, the pulse sequence may include information about a strength, a duration, and application timing of a pulse signal applied to the gradient magnetic field generator

The controller 30 may control a waveform generator (not shown) for generating a gradient wave, i.e., an electrical pulse according to a pulse sequence and a gradient amplifier (not shown) for amplifying the generated electrical pulse and transmitting the same to the gradient magnetic field generator 52. Thus, the controller 30 may control formation of a gradient magnetic field by the gradient magnetic field generator 52.

Furthermore, the controller 30 may control an operation of the RF coil unit 53. For example, the controller 30 may supply an RF pulse having a resonance frequency to the RF coil unit 30 that emits an RF signal toward the object, and receive an MR signal received by the RF control unit 53. In this case, the controller 30 may adjust emission of an RF signal and reception of an MR signal according to an operating mode by controlling an operation of a switch (e.g., a T/R switch) for adjusting transmitting and receiving directions of the RF signal and the MR signal based on a control signal.

The controller 30 may control a movement of the table 55 where the object is placed. Before MRI is performed, the controller 30 may move the table 55 according to which region of the object is to be imaged.

The controller 30 may also control the display 56. For example, the controller 30 control the on/off state of the display 56 or a screen to be output on the display 56 according to a control signal.

The controller 30 may be formed as an algorithm for controlling operations of the components in the MRI system 1, a memory (not shown) for storing data in the form of a program, and a processor for performing the above-described operations by using the data stored in the memory. In this case, the memory and the processor may be implemented as separate chips. Alternatively, the memory and processor may be incorporated into a single chip.

The controller 30 may include the processor 120 and the memory 130 of FIG. 1.

The operating unit 10 may control overall operations of the MRI system 1 and include an image processing unit 11, an input device 12, and an output device 13.

The image processing unit 11 may control the memory to store an MR signal received from the controller 30, and generate image data with respect to the object from the stored MR signal by applying an image reconstruction technique by using an image processor.

For example, if a k space (for example, also referred to as a Fourier space or a frequency space) of the memory is filled with digital data to complete k-space data, the image processing unit 11 may reconstruct image data from the k-space data by applying various image reconstruction techniques (e.g., by performing inverse Fourier transform on the k-space data) by using the image processor.

Furthermore, the image processing unit 11 may perform various signal processing operations on MR signals in parallel. For example, the image processor 62 may perform a signal process on a plurality of MR signals received by a multi-channel RF coil in parallel so as to rearrange the plurality of MR signals into image data. In addition, the image processing unit 11 may store not only the image data in the memory, or the controller 30 may store the same in an external server via a communication unit 60 as will be described below.

The input device 12 may receive, from the user, a control command for controlling the overall operations of the MRI system 1. For example, the input device 12 may receive, from the user, object information, parameter information, a scan condition, and information about a pulse sequence. The input device 12 may be a keyboard, a mouse, a track ball, a voice recognizer, a gesture recognizer, a touch screen, or any other input device.

The output device 13 may output image data generated by the image processing unit 11. The output device 13 may also output a user interface (UI) configured so that the user may input a control command related to the MRI system 1. The output device 13 may be formed as a speaker, a printer, a display, or any other output device.

Furthermore, although FIG. 12 shows that the operating unit 10 and the controller 30 are separate components, the operating unit 10 and the controller 30 may be included in a single device as described above. Furthermore, processes respectively performed by the operating unit 10 and the controller 30 may be performed by another component. For example, the image processing unit 11 may convert an MR signal received from the controller 30 into a digital signal, or the controller 30 may directly perform the conversion of the MR signal into the digital signal.

The MRI system 1 may further include a communication unit 60 and be connected to an external device (not shown) such as a server, a medical apparatus, and a portable device (e.g., a smartphone, a tablet PC, a wearable device, etc.) via the communication unit 60.

The communication unit 60 may include at least one component that enables communication with an external device. For example, the communication unit 60 may include at least one of a local area communication module (not shown), a wired communication module 61, and a wireless communication module 62.

The communication unit 60 may receive a control signal and data from an external device and transmit the received control signal to the controller 30 so that the controller 30 may control the MRI system 1 according to the received signal.

Alternatively, by transmitting a control signal to an external device via the communication unit 60, the controller 30 may control the external device according to the control signal.

For example, the external device may process data of the external device according to a control signal received from the controller 30 via the communication unit 60.

A program for controlling the MRI system 1 may be installed on the external device and may include instructions for performing some or all of the operations of the controller 30.

The program may be preinstalled on the external device, or a user of the external device may download the program from a server providing an application for installation. The server providing an application may include a recording medium having the program recorded thereon.

When a user manually positions the location of a region of interest into a scanner of an MRI apparatus, information about the location of the region of interest is provided to the user in real time, and thus a time period taken to position the region of interest may be reduced.

Programs stored in a server may be downloaded to another device or are downloadable. Computer-readable programs are downloadable in a remote data processing system so as to be used together with the remote data processing system by a computer readable recording medium.

The above-described embodiments of the present disclosure may be embodied in form of a computer-readable recording medium for storing computer executable command languages and data. The command languages may be stored in form of program codes and, when executed by a processor, may perform a certain operation by generating a certain program module. Also, when executed by a processor, the command languages may perform certain operations of the disclosed embodiments.

According to an embodiment, a computer program product including a computer-readable recording medium capable of storing computer-readable programs is provided. Also, when executed by a processor, the computer-readable programs may perform operations or methods of the disclosed embodiments.

According to an embodiment, a computer program product, and a system including an MR image display apparatus that performs operations according to computer programs recorded in the computer program product are provided. The computer program product may store the computer-readable programs that perform operations or methods of the disclosed embodiments. The MR image display apparatus may download the computer programs recorded in the computer program product and may perform the computer programs.

While embodiments of the present disclosure have been particularly shown and described with reference to the accompanying drawings, it will be understood by those of ordinary skill in the art that various changes in form and details may be made therein without departing from the spirit and scope of the inventive concept as defined by the appended claims. The disclosed embodiments should be considered in descriptive sense only and not for purposes of limitation.

1. A magnetic resonance (MR) display apparatus comprising:
a display;
a processor; and
a memory connected to the processor, wherein, the memory stores instructions executed by the processor for:
receiving an input regarding a region of interest of an object;
obtaining a positioning image for determining a location of the region of interest within a scanner of a magnetic resonance imaging (MRI) apparatus; and
displaying, on the display, whether the location of the region of interest included in the positioning image corresponds to a location of an isocenter of the scanner.

2. The MR image display apparatus of claim 1, wherein the memory further stores an instruction executed by the processor for obtaining the positioning image while a location of the object within the scanner is being changed.

3. The MR image display apparatus of claim 1, wherein
the memory further stores an instruction executed by the processor for overlapping the positioning image with guide information and displaying a result of the overlapping, when a location of a predetermined spot on the region of interest is within a first distance from the isocenter, and
the guide information includes at least one base line representing the location of the isocenter, and location information of the region of interest.

4. The MR image display apparatus of claim 3, wherein the location information of the region of interest includes at least one of location information of the predetermined spot with respect to the isocenter and a direction in which the object is to move.

5. The MR image display apparatus of claim 4, wherein the location information of the region of interest includes a degree to which a location of the predetermined spot deviates from the isocenter.

6. The MR image display apparatus of claim 1, wherein the memory further stores an instruction executed by the processor for repeating the obtaining of the positioning image and the displaying of, on the display, whether the location of the region of interest included in the positioning image corresponds to the location of the isocenter.

7. The MR image display apparatus of claim 1, wherein the memory further stores an instruction executed by the processor for stopping the obtaining of the positioning image when the location of the object within the scanner is not changed for a predetermined time period or when it is determined that the location of the region of interest corresponds to the location of the isocenter.

8. The MR image display apparatus of claim 1, wherein the memory further stores an instruction executed by the processor for displaying, by using at least one light-emitting device, whether the location of the region of interest corresponds to the location of the isocenter.

9. The MR image display apparatus of claim 8, wherein the memory further stores an instruction executed by the processor for displaying, by using the at least one light-emitting device, at least one of location information of the predetermined spot on the region of interest with respect to the isocenter, a direction in which the object is to move, and a degree to which the location of the predetermined spot deviates from the location of the isocenter.

10. A method of displaying a magnetic resonance (MR) image, the method comprising:
receiving an input regarding a region of interest of an object,
obtaining a positioning image for determining a location of the region of interest within a scanner of a magnetic resonance imaging (MRI) apparatus; and
displaying, on a display, whether the location of the region of interest included in the positioning image corresponds to a location of the isocenter of the scanner.

11. The method of claim 10, wherein the obtaining of the positioning image comprises obtaining the positioning image while a location of the object within the scanner is being changed.

12. The method of claim 10, further comprising overlapping the positioning image with guide information and displaying a result of the overlapping, when a location of a predetermined spot on the region of interest is within a first distance from the isocenter,
wherein the guide information includes at least one base line representing the location of the isocenter, and location information of the region of interest.

13. The method of claim 12, wherein the location information of the region of interest includes at least one of location information of the predetermined spot with respect to the isocenter and a direction in which the object is to move.

14. The method of claim 13, wherein the location information of the region of interest includes a degree to which a location of the predetermined spot deviates from the isocenter.

15. The method of claim 10, wherein the obtaining of the positioning image and the displaying are repeated.

16. The method of claim 10, further comprising stopping the obtaining of the positioning image when the location of the object within the scanner is not changed for a predetermined time period.

17. The method of claim 10, further comprising displaying, by using at least one light-emitting device, whether the location of the region of interest corresponds to the location of the isocenter.

18. The method of claim 17, further comprising displaying, by using the at least one light-emitting device, at least one of location information of the predetermined spot on the region of interest with respect to the isocenter, a direction in which the object is to move, and a degree to which the location of the predetermined spot deviates from the location of the isocenter.

19. A computer-readable recording medium having recorded thereon a computer program for executing the method of claim 10.
